(19) United States
(12) Patent Application Publication
(10) Pub. No.: US 2026/0325408 A1
(43) Pub. Date: Oct. 22, 2026

(54) Fluoride-Free Nano-Hydroxyapatite Dental Varnish for Enamel Remineralization

(71) Applicant: ENML Group LLC, Niceville, FL (US)

(72) Inventors: Kyle Schroeder, Miramar, FL (US); Jordan Harper, Niceville, FL (US)

(73) Assignee: ENML Group LLC, Niceville, FL (US)

(21) Appl. No.: 19/571,102

(22) Filed: Mar. 18, 2026

Related U.S. Application Data
(60) Provisional application No. 63/791,399, filed on Apr. 19, 2025.

(51) Int. Cl. A61K 6/20; A61K 6/17; A61K 6/69

(52) U.S. Cl. CPC A61K 6/20; A61K 6/17; A61K 6/69

(57) ABSTRACT

A fluoride-free dental varnish and application method for remineralizing enamel and reducing dentin hypersensitivity is provided wherein, after professional dental cleaning and drying, a unit-dose or brush-applied varnish comprising nano-hydroxyapatite particles (D50<100 nm, 5-15 wt %), an ammonio methacrylate copolymer film-former (10-30 wt %, e.g., Eudragit RS PO), ethanol as the volatile solvent (50-80 wt %), and optional flavoring oil (0.1-2 wt % peppermint) is applied to tooth surfaces; the wet film dries tack-free within seconds to minutes to form a continuous, water-insoluble yet ion-permeable polymer coating (dry thickness 1-50 μm) that retains nano-hydroxyapatite, sustains release of calcium and phosphate ions (≥0.05 mg/cm2 over 4 h in simulated saliva), and physically occludes dentin tubules to reduce hypersensitivity; the varnish is free of added fluoride and rosin/colophony, remains adherent for hours to days, and may be reapplied at intervals of one week to six months.

### BACKGROUND OF THE INVENTION

Dental hard tissues are continually challenged by acidic demineralization from dietary acids and bacterial metabolism, leading to enamel erosion, caries progression, and, when dentin becomes exposed, painful hypersensitivity due to fluid movement within open dentinal tubules. Fluoride-based therapies have long been the predominant means of promoting remineralization and reducing sensitivity, but interest in fluoride-free, biomimetic alternatives has grown in response to concerns about overexposure and the desire for restorative approaches that more closely replicate natural mineral phases. Nanoscale hydroxyapatite particles have emerged in the literature as a promising bioactive agent because their size and chemical similarity to tooth mineral facilitate deposition and integration with enamel apatite and can contribute to tubule occlusion. Effective clinical performance of such actives depends not only on intrinsic bioactivity but also on the delivery medium: film-forming polymers that adhere to tooth surfaces can prolong contact time, protect deposited material from immediate removal, and influence ion exchange and tubule sealing. Consequently, research and formulation efforts have focused on combining biomimetic mineral particles with durable, biocompatible film-formers to enhance remineralization efficacy and provide sustained relief from dentin hypersensitivity.

### SUMMARY OF THE INVENTION

In one aspect, a method for remineralizing tooth enamel and reducing dentin hypersensitivity in a subject comprises, after a professional dental cleaning, drying exposed tooth surfaces and applying to the dried tooth surfaces an amount effective to coat them with a fluoride-free varnish composition containing nano-hydroxyapatite particles dispersed in ethyl alcohol together with an ammonio methacrylate copolymer film-former and optionally a flavoring oil. The ethyl alcohol is allowed to evaporate to form on the tooth surfaces a continuous adherent polymer film that retains the nano-hydroxyapatite, and the film is maintained on the tooth surfaces for a dwell time sufficient to release calcium and phosphate ions from the nano-hydroxyapatite and to at least partially occlude dentin tubules.

In yet another aspect, a fluoride-free dental varnish composition comprises nanoparticulate hydroxyapatite dispersed in an ethanol solution of an ammonio methacrylate copolymer film-former. The ethanol serves as a volatile solvent to permit brushing application and rapid setting by evaporation. The composition optionally includes a flavoring oil. Upon application to cleaned tooth surfaces and drying, the composition forms a continuous adherent coating that releases calcium and phosphate ions to promote enamel remineralization and physically occludes dentin tubules to reduce hypersensitivity.

Advantages of one implementation may include one or more of the following:
Provides a fluoride-free remineralization option, addressing concerns about fluoride overexposure while offering a clinically useful alternative.
Incorporates nanoparticulate hydroxyapatite that is chemically and structurally similar to tooth mineral, promoting deposition, nucleation, and integration with enamel apatite.
Nano-scale particles offer high surface area for enhanced dissolution and release of calcium and phosphate ions, supporting enamel remineralization and repair.
The ammonio methacrylate copolymer film-former produces a continuous, adherent coating on cleaned tooth surfaces that prolongs contact time of the active particles with enamel and dentin.
The polymer film protects deposited hydroxyapatite from immediate mechanical removal (e.g., by saliva or brushing), enabling sustained ion exchange and longer dwell time.
Rapid setting by ethanol evaporation enables convenient chairside application with short procedure time and immediate film formation.
Physical entrapment and deposition of nano-hydroxyapatite within the polymer film facilitates partial or complete occlusion of exposed dentin tubules, providing immediate and sustained reduction in dentin hypersensitivity.
The composition can form a thin, transparent coating that preserves tooth appearance and is compatible with routine oral function.
Optional inclusion of a flavoring oil improves patient acceptability and masking of solvent sensory effects without compromising film formation.
The formulation is adaptable for concentration and particle-size adjustments to tailor ion-release kinetics and tubule-occluding performance.
Use after professional cleaning maximizes surface contact and bonding of the film to enamel and exposed dentin, improving efficacy.
Non-invasive, topical application may reduce the need for more aggressive restorative interventions by promoting remineralization and protecting vulnerable surfaces.
Components are compatible with common dental practice workflows and equipment, facilitating adoption for chairside use or professional application.
The varnish architecture enables controlled, sustained delivery of bioavailable calcium and phosphate from a biomimetic source while minimizing systemic exposure.

Each of the foregoing advantages may be realized alone or in combination, depending on formulation parameters and clinical use conditions.

### BRIEF DESCRIPTION OF DRAWINGS

Turning now descriptively to the drawings, in which similar reference characters denote similar elements throughout the several views, the figures illustrate the electronic book of the present invention. With regard to the reference numerals used, the following numbering is used throughout the various drawing figures.

FIG. 1 illustrates a flowchart for applying a fluoride-free nano-hydroxyapatite dental varnish: drying teeth after cleaning, brushing on the composition, evaporating ethanol to form a polymer film, and maintaining the film to release ions and occlude dentin tubules.

### DETAILED DESCRIPTION OF THE INVENTION

The following discussion describes in detail one embodiment of the invention (and several variations of that embodiment). This discussion should not be construed, however, as limiting the invention to those particular embodiments, practitioners skilled in the art will recognize numerous other embodiments as well. For definition of the complete scope of the invention, the reader is directed to appended claims.

In the following paragraphs, the present invention will be described in detail by way of example with reference to the attached drawings. Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

This invention now will be described more fully hereinafter with reference to the accompanying drawings, in which exemplary embodiments are shown. Various embodiments are now described with reference to the drawings, wherein such as reference numerals are used to refer to such as elements throughout. In the following description, for purposes of explanation, numerous specific details are set forth in order to provide a thorough understanding of one or more embodiments. It may be evident, however, that such embodiment(s) may be practiced without these specific details. In other instances, well-known structures and devices are shown in block diagram form in order to facilitate describing one or more embodiments.

This invention may, however, be embodied in many different forms and should not be construed as limited to the embodiments set forth herein. These embodiments are provided so that this disclosure will be thorough and complete and will fully convey the scope of the invention to those of ordinary skill in the art. Moreover, all statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

Thus, for example, it will be appreciated by those of ordinary skill in the art that the diagrams, schematics, illustrations, and the such as represent conceptual views or processes illustrating systems and methods embodying this invention. The functions of the various elements shown in the figures may be provided through the use of dedicated hardware as well as hardware capable of executing associated software. Similarly, any switches shown in the figures are conceptual only. Their function may be carried out through the operation of program logic, through dedicated logic, through the interaction of program control and dedicated logic, or even manually, the particular technique being selectable by the entity implementing this invention. Those of ordinary skill in the art further understand that the exemplary hardware, software, processes, methods, and/or operating systems described herein are for illustrative purposes and, thus, are not intended to be limited to any particular named manufacturer.

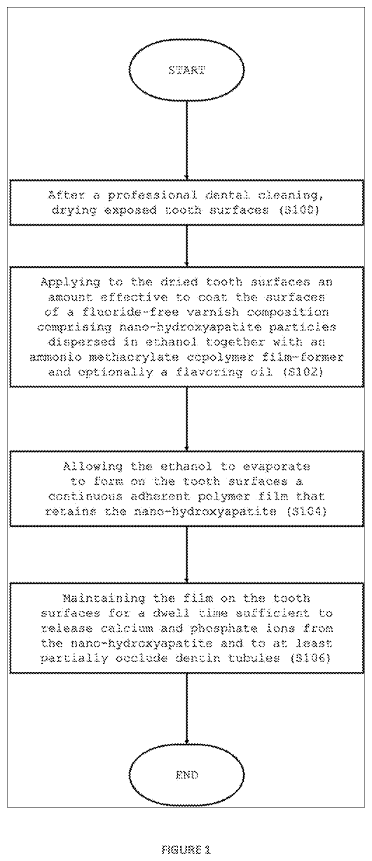

FIG. 1 illustrates a flowchart for applying a fluoride-free nano-hydroxyapatite dental varnish: drying teeth after cleaning, brushing on the composition, evaporating ethanol to form a polymer film, and maintaining the film to release ions and occlude dentin tubules.

Following a professional dental cleaning, the teeth are dried and coated with a fluoride-free varnish comprising nano-hydroxyapatite dispersed in ethanol together with an ammonio methacrylate copolymer film-former (optionally containing flavoring oil). The ethanol evaporates to leave an adherent polymer film that retains the nano-hydroxyapatite on the tooth surface for a sufficient period to release calcium and phosphate ions and to at least partially occlude dentin tubules.
This dependent claim narrows the method to using a varnish that contains no fluoride.
In other words, the composition applied in the procedure is formulated without any fluoride compounds or ions.

The method uses an ammonio methacrylate copolymer as the film-forming component. It creates a continuous polymer film. The film remains adhered to the tooth surfaces.

The method of claims 1-3 can use a varnish that also contains a flavoring oil. This oil is an optional additive to improve taste and patient acceptability.

After application, the ethanol solvent evaporates at room temperature.
This evaporation leaves behind a retained polymer film on the surface.

The coating is retained on the teeth for a period sufficient to permit release of calcium and phosphate ions. The released ions deposit, resulting in at least partial blockage of the dentin tubules.
The method is intended to reduce or treat dentin hypersensitivity.
It achieves this by occluding the dentin tubules.
After application, the ethanol evaporates, leaving a continuous, adherent polymer film on teeth that retains the nano-hydroxyapatite.

FIG. 1 illustrates a sequential dental treatment process in which, after a professional dental cleaning, the exposed tooth surfaces are dried (S100), an amount effective to coat the dried tooth surfaces of a fluoride-free varnish composition comprising nano-hydroxyapatite particles dispersed in ethanol together with an ammonio methacrylate copolymer film-former and optionally a flavoring oil is applied (S102), the ethanol is allowed to evaporate to form on the tooth surfaces a continuous adherent polymer film that retains the nano-hydroxyapatite (S104), and the film is maintained on the tooth surfaces for a dwell time sufficient to release calcium and phosphate ions from the nano-hydroxyapatite and to at least partially occlude dentin tubules (S106). The applied varnish composition at S102 is fluoride-free and provides the nano-hydroxyapatite dispersed in ethanol and the film-former; evaporation at S104 converts the application into a continuous, adherent film that entraps the nano-hydroxyapatite; and the maintained film at S106 enables ion release and tubule occlusion. The process proceeds sequentially S100→S102→S104→S106, with each step enabling the next.

The reference label S100 denotes the step of, following a professional dental cleaning, drying the exposed tooth surfaces prior to application of the varnish.

Step S104 involves allowing the ethanol to evaporate so that the ammonio methacrylate copolymer coalesces into a continuous polymer matrix on the tooth surfaces. As the ethanol volatilizes, the polymer film adheres to enamel and dentin and mechanically entraps the dispersed nano-hydroxyapatite particles within the matrix. The resulting continuous adherent film retains the nanoparticles against the tooth surface, provides a controlled interface from which calcium and phosphate ions can be released during the dwell time, and contributes to physical occlusion of dentin tubules.

In one aspect, a method for remineralizing tooth enamel and reducing dentin hypersensitivity comprises, after a professional dental cleaning, drying exposed tooth surfaces and applying to the dried tooth surfaces an amount effective to coat them with a fluoride-free varnish composition comprising nanoparticulate hydroxyapatite dispersed in ethyl alcohol together with an ammonio methacrylate copolymer film-former and optionally a flavoring oil. The ethyl alcohol is permitted to evaporate to form on the tooth surfaces a continuous adherent polymer film that retains the nanoparticulate hydroxyapatite, and the resulting film is maintained on the tooth surfaces for a dwell time sufficient to release calcium and phosphate ions from the nanoparticulate hydroxyapatite and to at least partially occlude dentin tubules, thereby promoting enamel remineralization and reducing dentin hypersensitivity.

Reference label S106 refers to maintaining the formed polymer film on the tooth surfaces for a dwell time sufficient to permit release of calcium and phosphate ions from the nanoparticulate hydroxyapatite and to achieve at least partial occlusion of dentin tubules; the dwell time is selected to allow the ion release and physical deposition processes to proceed to an extent effective to contribute to enamel remineralization and reduction of dentin hypersensitivity.

The varnish is applied with a microbrush, gloved finger, or tray insert in a volume ranging from 0.05 mL to 0.5 mL per arch, sufficient to wet and visibly coat all intended tooth surfaces including exposed dentin. Allowing the ethanol to evaporate for 30 seconds to 5 minutes under ambient conditions, optionally assisted by a gentle air stream, yields on the tooth surfaces a continuous, adherent polymer film that retains the nano-hydroxyapatite. The film typically has a dry thickness of 2-15 μm and conforms to pits, fissures, and dentin tubule openings. The ammonio methacrylate copolymer confers pH-independent mucoadhesion and moisture resistance, maintaining intimate contact with enamel and dentin.

The film is maintained on the tooth surfaces for a dwell time sufficient to release calcium and phosphate ions from the nano-hydroxyapatite and to at least partially occlude dentin tubules. In some embodiments, the patient is instructed to avoid eating, drinking, and toothbrushing for at least 30 minutes, preferably 2-6 hours, more preferably 4 hours or overnight. During dwell, saliva and plaque fluid promote controlled dissolution of surface nano-hydroxyapatite, supplying Ca2+ and PO43− that deposit within enamel microdefects and within or across dentin tubule orifices. In vitro testing shows ion release of at least 0.1-10 mg/L Ca2+ over 1-24 hours, and scanning electron microscopy demonstrates 50% to 90% tubule orifice coverage after a 4-hour dwell. The film naturally erodes with normal oral hygiene over 12-72 hours, leaving residual hydroxyapatite integrated with the surface.

An example composition comprises 10 wt % nano-hydroxyapatite, 20 wt % ammonio methacrylate copolymer, 69 wt % ethanol (USP 95%), and 1 wt % peppermint oil. An 0.1 mL application per quadrant, dried 1-2 minutes and maintained for at least 4 hours, provides enamel remineralization and measurable reduction in hypersensitivity via partial or complete dentin tubule occlusion.

In embodiments, the ammonio methacrylate copolymer is Eudragit RS PO, a quaternary-ammonium-containing copolymer of ethyl acrylate, methyl methacrylate, and trimethylammonioethyl methacrylate chloride that is swellable and permeable but insoluble in water and readily soluble in ethanol. Eudragit RS PO provides a cationic, pH-independent, film-forming matrix that adheres to enamel and dentin and retains nano-hydroxyapatite (nano-HA) at the tooth surface while permitting controlled diffusion of saliva and ions. The varnish is prepared by dissolving Eudragit RS PO in anhydrous or denatured ethanol at 10-30 wt % polymer relative to total composition, mixing until a clear solution forms. Nano-HA powder having a primary particle size preferably below 100 nm, more preferably 20-80 nm with a narrow distribution, is incrementally dispersed into the polymer solution at 5-15 wt % using intensive shear mixing and, as needed, probe sonication to minimize agglomerates. The ethanol content is 50-80 wt % to provide brushable viscosity and rapid solvent flash-off. A flavoring oil such as peppermint oil is optionally included at 0.1-1.5 wt % to improve patient acceptance. The composition is fluoride-free and can be filled into unit-dose applicators under inert gas to limit moisture uptake.

On application to professionally cleaned and dried teeth, a thin layer is painted with a microbrush or gloved finger in an amount sufficient to wet and cover exposed surfaces. Ethanol evaporates within seconds to a few minutes at oral temperatures to yield a continuous adherent film containing dispersed nano-HA. Typical dry film thickness is 2-50 μm depending on solids loading and stroke count. The cationic quaternary groups of Eudragit RS PO promote electrostatic interaction with negatively charged hydroxyapatite on enamel and with exposed collagen/mineral in dentin, enhancing mechanical retention over several hours. The polymer's permeability allows saliva exchange while resisting immediate wash-off, providing a dwell period of at least 1-4 hours, optionally up to overnight use, during which nano-HA releases calcium and phosphate ions and physically lodges within and across dentin tubule openings to effect partial occlusion and sensitivity relief. The film gradually erodes through normal oral function and is removable by routine toothbrushing.

An illustrative formulation comprises, by weight, 10% nano-HA, 20% Eudragit RS PO, 69.5% ethanol, and 0.5% peppermint oil. Manufacturing includes charging ethanol, dissolving the polymer at ambient temperature with overhead stirring until homogeneous, adding nano-HA in portions under rotor-stator shear at approximately 3,000-6,000 rpm, followed by 1-5 minutes of ultrasonic treatment to reduce agglomeration, and finally adding peppermint oil with gentle mixing. The varnish exhibits a workable viscosity of 100-2,000 mPa·s at 25 °C, remains physically stable for at least six months in sealed containers, and forms a clear to slightly opalescent film on teeth upon solvent evaporation.

The total solids content is typically 15-47 wt %, the remainder being ethanol, producing a pourable, brushable varnish with a viscosity at 25 °C in the range of 50-1,500 mPa·s, depending on polymer level and particle loading. The composition is fluoride-free and excludes rosin/colophony and other known sensitizers. Optional stabilizers or anti-settling aids are unnecessary; however, up to 0.2 wt % fumed silica can be incorporated without departing from the described ranges.

A representative manufacturing process comprises dissolving the ammonio methacrylate copolymer in ethanol under mechanical stirring at room temperature until a clear solution forms. Nano-hydroxyapatite is incrementally added to the polymer solution and dispersed by vigorous shear mixing followed by ultrasonic treatment to achieve a mean hydrodynamic diameter below 200 nm. Peppermint oil is incorporated last with gentle mixing to minimize air entrapment. The varnish is passed through a 50-100 μm screen, filled into unit-dose applicators, and sealed. The product remains stable for at least 12 months at 15-30 °C when stored in opaque, sealed containers.

In embodiments, a varnish composition comprises nano-hydroxyapatite particles dispersed in ethanol and an ammonio methacrylate copolymer film-former, optionally with a flavoring oil, wherein the varnish composition is free of fluoride compounds. “Fluoride-free” means that no fluoride-releasing or fluoride-containing ingredients (including NaF, SnF2, Na2PO3F, amine fluorides, or fluorosilanes) are deliberately added, and residual fluoride, if present as an unavoidable impurity, is below 50 ppm, preferably below 10 ppm, as measured by ion-selective electrode or ion chromatography.

Nano-hydroxyapatite (HA) has a primary particle size from 10 to 200 nm, preferably 20 to 100 nm, and is rod-like, plate-like, or spherical. The HA is stoichiometric or calcium-rich and is optionally surface-treated with biocompatible dispersants such as trace citrate or phosphate to improve suspension stability without blocking ion release. HA solids content ranges from 2 to 20 wt %, preferably 5 to 15 wt % of the composition.

A representative preparation comprises dispersing the polymer into ethanol under agitation to form a clear solution, adding nano-HA while applying intensive shear mixing or probe sonication to produce a uniform suspension, and introducing the flavor oil last. The dispersion has a viscosity suitable for application to teeth with a brush or swab and remains physically stable for at least six months under ambient storage conditions.

The varnish is prepared by dissolving the copolymer in anhydrous ethanol with stirring at ambient temperature, adding flavor oil, and dispersing nano-HA using vigorous shear mixing and, if desired, brief ultrasonication or bead-milling to minimize agglomeration. Target solids are 20-45 wt % to balance brushability and rapid set. Viscosity is adjustable between 50 and 1500 mPa·s at 25 °C to suit different applicators.

The coating gradually wears off with normal oral hygiene over hours to days. The method can be repeated at intervals, for example weekly to quarterly, and is compatible with conventional prophylaxis procedures, including polishing pastes free of oil residues that could interfere with film adhesion.

The water-insoluble character is evidenced by a mass loss in water at 37 °C of less than 1 wt % over 4 hours and by maintenance of film integrity during normal salivary exposure. In certain implementations the film exhibits saline uptake of 2-15 wt % and a calcium ion permeability coefficient ranging from 1×10\hat{ }−8 to 1×10\hat{ }−6 cm\hat{ }2/s as measured in a Franz diffusion cell. Typical dry film thickness produced by a single brush application is 2-30 μm, more typically 5-20 μm, which balances mechanical durability with ion transport. The coating remains adherent during the dwell period of S106, commonly 4-12 hours, and naturally wears off with routine hygiene within 1-3 days.

The ion-permeable, water-insoluble behavior is tunable by formulation variables including polymer grade (RS alone or blends of RS with the more permeable RL grade, e.g., RS:RL weight ratios of 100:0 to 30:70), total solids content, nano-hydroxyapatite loading, and drying conditions. Increasing the polymer fraction and film thickness reduces ion flux, whereas increased RL content or reduced film thickness raises ion flux. Flavoring oil at ≤2 wt % can slightly plasticize the matrix and enhance permeability without compromising water insolubility. In certain embodiments the varnish contains 10-30 wt % ammonio methacrylate copolymer, 5-15 wt % nano-hydroxyapatite, and 50-80 wt % ethanol, resulting in rapid setting and a continuous, non-tacky film.

Sustained release is demonstrated by immersing coated enamel or dentin specimens in artificial saliva maintained at 37 °C, with periodic sampling for Ca2+ and PO 4\hat{ }3− using ICP-OES or ion-selective electrodes. Representative release profiles exhibit an initial diffusion-controlled phase followed by near zero-order delivery, yielding cumulative calcium release in the range of 0.05 to 2 mg/cm\hat{ }2 over periods of 2 to 24 hours, during which the nano-hydroxyapatite remains on the surface. The steady liberation of ions supports enamel remineralization and progressive physical occlusion of dentin tubules, while the persistent, water-insoluble film protects the particles from immediate wash-off in the oral environment.

The depth of physical occlusion obtainable with the described compositions is at least 1 μm under typical clinical conditions, and in some embodiments at least 2 μm, 5 μm, or 10 μm. Occlusion depth can be increased by applying multiple thin coats separated by brief evaporation intervals, by using nHA concentrations between 5 and 15 wt % relative to the total composition, by selecting copolymer levels between 10 and 30 wt % to optimize viscosity and film strength, and by maintaining a dwell time of at least 4 hours before vigorous oral hygiene. The ammonio methacrylate copolymer provides a cationic, moisture-tolerant, pH-independent matrix that adheres to dentin, bridges across tubule orifices, and resists rapid dissolution so that the nHA plugs remain anchored while ions are released.

Occlusion depth and extent can be verified on extracted dentin or in vivo samples by preparing cross-sections and imaging with scanning electron microscopy or focused ion beam-SEM to measure the axial length of nHA-containing material inside tubules. In additional embodiments, dye penetration or fluid conductance testing shows a reduction in dentin permeability of at least 30%, 50%, 70%, or 90% relative to untreated controls, correlating with measured occlusion depth. Energy-dispersive X-ray spectroscopy can confirm calcium- and phosphate-rich deposits inside tubules attributed to nHA and newly formed apatite. The occluding structures formed by the dispersed nHA are physically confined and mechanically stabilized by the polymer film, providing immediate sensitivity relief and resistance to dislodgement by gentle rinsing, with gradual wear occurring over hours to days under normal oral conditions.

The applying is performed using a unit-dose applicator including a brush or microbrush. In representative devices a sealed ampoule, blister, or vial contains 0.1-1.0 mL of the varnish and is provided with an integral brush or microbrush for single-patient use to maintain sterility and dose control. The clinician expresses the contents and paints the varnish onto cleaned tooth surfaces, including cervical and root areas, moving the brush along the gingival margins and exposed dentin to achieve a uniform wet coating of approximately 5-50 micrometers thickness. Multiple thin coats can be applied to increase hydroxyapatite loading.

The nano-HA preferably has a primary particle size less than 100 nm, more typically 20 to 80 nm, with a surface area in the range of 50 to 200 m2/g, providing enhanced penetration into surface defects and dentin tubules. The copolymer contains quaternary ammonium groups that afford a moisture-tolerant, adherent, and continuous film in the oral environment. Ratios of polymer to nano-HA range from 1:1 to 6:1 by weight to balance film integrity and particle loading.

The method can be repeated to maintain benefits, with reapplication intervals ranging from 1 week to 6 months depending on patient sensitivity, caries risk, and professional judgment. In some implementations, weekly applications are used for an initial intensive phase followed by monthly or quarterly maintenance. Variations include use of other ammonio methacrylate copolymer grades that provide similar film-forming properties, minor water content up to 10% to adjust viscosity, and alternative flavoring oils, while remaining fluoride-free.

The composition is free of water, plasticizers, rosin, colophony, and fluoride actives, except for incidental impurities and trace stabilizers that do not materially change the varnish character. Solids content and polymer-to-particle ratio are selected to provide an application viscosity in the range 50 to 800 mPa·s at 25 °C, permitting even coating by brush, micro-applicator, gloved finger, or tray insert. Exemplary ratios include polymer: nano-hydroxyapatite from 1:0.2 to 1:2 by weight.

A representative preparation comprises charging a vessel with the required mass of ethanol, slowly adding Eudragit RS PO under moderate agitation until fully dissolved, introducing nano-hydroxyapatite as a pre-wetted slurry in a portion of ethanol, and dispersing by intense shear mixing or probe sonication to a mean particle size below 100 nm. Flavoring oil is blended last. The batch is degassed, filtered through a 50-100 μm mesh, and filled under dry conditions into unit-dose applicators or amber bottles. The composition exhibits stability at ambient temperature for at least six months when stored tightly sealed.

In vitro, coated bovine enamel or dentin coupons immersed in artificial saliva at 37 °C show sustained Ca2+ and PO43− release for 2-12 hours and ≥50% reduction in open dentin tubule area by SEM compared with untreated controls. The composition remains stable for at least 12 months at 20-25 °C in sealed containers with minimal sedimentation that readily re-disperses upon gentle agitation.

Compositions are free of fluoride, rosin, colophony, plasticizers, and colorants, minimizing allergenicity while maintaining effective adhesion and ion release. Optional excipients, such as trace amounts of silica anti-settling agents or ethanol-compatible preservatives, can be included without departing from the essence of the system. The varnish is supplied as a single-use applicator comprising a brush or foam tip preloaded with 0.1-1.0 mL of composition, packaged in a sealed foil pouch or vial to limit ethanol loss; a typical adult full-mouth dose is 0.3-0.5 mL. The device facilitates hygienic, uniform delivery, consistent film formation, and disposal after one patient use.

The dried coating provides sustained calcium ion release of at least 0.05 mg per cm² over 4 hours in simulated saliva. One test method employs enamel or inert glass coupons having an exposed area of 1.0±0.1 cm² coated as described and dried for 5 minutes. Each coupon is immersed in 10 mL of calcium-free simulated saliva at pH 6.8-7.0 (37 °C, gentle agitation 60 rpm), prepared as a modified Fusayama-Meyer medium omitting calcium salts and optionally containing KCl 0.4 g/L, NaCl 0.4 g/L, KH₂PO₄ 0.69 g/L, urea 1.0 g/L, and buffer to pH 6.8. Aliquots are withdrawn at 0.5, 1, 2, and 4 hours and analyzed for Ca²⁺ by ICP-OES or ion-selective electrode using external standards. Across a minimum of five samples, the cumulative calcium released at 4 hours is not less than 0.05 mg/cm², typically 0.06-0.25 mg/cm², with concomitant phosphate release of 0.03-0.20 mg/cm². Films remain adherent throughout testing without visible delamination.

Scanning electron microscopy of dentin specimens treated as described demonstrates partial to substantial tubule occlusion after 4 hours, commonly exceeding 50% occluded area, and the occluding deposits contain calcium and phosphate consistent with hydroxyapatite. The film wears off naturally over subsequent hours or days with normal hygiene. Packaging is provided in single-use, ethanol-tight applicators to maintain stability for at least 18 months at room temperature.

Various modifications and alterations of the invention will become apparent to those skilled in the art without departing from the spirit and scope of the invention, which is defined by the accompanying claims. It should be noted that steps recited in any method claims below do not necessarily need to be performed in the order that they are recited. Those of ordinary skill in the art will recognize variations in performing the steps from the order in which they are recited. In addition, the lack of mention or discussion of a feature, step, or component provides the basis for claims where the absent feature or component is excluded by way of a proviso or similar claim language.

While various embodiments of the present invention have been described above, it should be understood that they have been presented by way of example only, and not of limitation. The various diagrams may depict an example architectural or other configuration for the invention, which is done to aid in understanding the features and functionality that may be included in the invention. The invention is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the present invention. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the invention, whether or not such embodiments are described and whether or not such features are presented as being a part of a described embodiment. Thus the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the such as; the term “example” is used to provide exemplary instances of the item in discussion, not an exhaustive or limiting list thereof; the terms “a” or “an” should be read as meaning “at least one,” “one or more” or the such as; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Hence, where this document refers to technologies that would be apparent or known to one of ordinary skill in the art, such technologies encompass those apparent or known to the skilled artisan now or at any time in the future.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the invention may be described or claimed in the singular, the plural is contemplated to be within the scope thereof unless limitation to the singular is explicitly stated.

The presence of broadening words and phrases such as “one or more,” “at least,” “but not limited to” or other such as phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, may be combined in a single package or separately maintained and may further be distributed across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

It is to be understood that the above description is intended to be illustrative, and not restrictive. For example, the above-described embodiments (and/or aspects thereof) may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects. The Abstract of the Disclosure is provided to comply with 37 C.F.R. &sect; 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, various features may be grouped together to streamline the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

1. A method for remineralizing tooth enamel and reducing dentin hypersensitivity in a subject, comprising:
after a professional dental cleaning, drying exposed tooth surfaces;
applying to the dried tooth surfaces an amount effective to coat the exposed surfaces of the exposed tooth surfaces in a fluoride-free varnish composition comprising nano-hydroxyapatite particles dispersed in ethanol together with an ammonio methacrylate copolymer film-former and optionally a flavoring oil;
allowing the ethanol to evaporate to form on the tooth surfaces a continuous adherent polymer film that retains the nano-hydroxyapatite;
maintaining the film on the tooth surfaces for a dwell time sufficient to release calcium and phosphate ions from the nano-hydroxyapatite and to at least partially occlude dentin tubules

2. The method of claim 1, wherein the ammonio methacrylate copolymer is Eudragit RS PO.

3. The method of claim 1, wherein the varnish composition comprises, by weight, 5-15% nano-hydroxyapatite, 10-30% ammonio methacrylate copolymer, 50-80% ethanol, and 0.1-2% peppermint oil.

4. The method of claim 1, wherein the varnish composition is free of fluoride compounds.

5. The method of claim 1, wherein the nano-hydroxyapatite particles have a median particle diameter less than 100 nanometers.

6. The method of claim 1, wherein applying the varnish produces a wet film that dries to a tack-free state within 10 seconds to 5 minutes by ethanol evaporation.

7. The method of claim 1, wherein the formed film has a dry thickness between 1 and 50 micrometers.

8. The composition of claim 7, wherein the flavoring oil comprises mentha piperita oil present at 0.1 to 2 percent by weight of the total composition.

9. The method of claim 1, wherein the dwell time is at least 4 hours.

10. The method of claim 1, further comprising instructing the subject to avoid eating, drinking, or toothbrushing during the dwell time.

11. The method of claim 1, wherein the polymer film is water-insoluble and ion-permeable such that calcium and phosphate ions are released in a sustained manner in the oral environment.

12. The method of claim 1, wherein the nano-hydroxyapatite physically occludes exposed dentin tubules to a depth of at least 1 micrometer.

13. The method of claim 1, wherein the applying is performed using a unit-dose applicator including a brush or microbrush.

14. The method of claim 1, wherein the coating remains adherent on the teeth for 2 to 48 hours and subsequently wears off naturally with normal oral hygiene.

15. The method of claim 1, wherein the varnish composition lacks rosin or colophony.

16. The method of claim 1, further comprising repeating application at intervals of 1 week to 6 months to maintain enamel strengthening and sensitivity relief.

17. A fluoride-free dental varnish composition comprising nano-hydroxyapatite particles dispersed in an ethanol solution of an ammonio methacrylate copolymer film-former, the ethanol serving as a volatile solvent to allow brushing application and rapid setting by evaporation, and optionally a flavoring oil, wherein upon application to cleaned tooth surfaces and drying the composition forms a continuous adherent coating configured to release calcium and phosphate ions for enamel remineralization and to physically occlude dentin tubules for hypersensitivity reduction.

18. The composition of claim 17, wherein the nano-hydroxyapatite particles have a D50 primary particle size of less than 100 nm.

19. The composition of claim 17, wherein the nano-hydroxyapatite is present at 5 to 40 percent by weight of the total composition

20. The composition of claim 17, wherein the ammonio methacrylate copolymer is a pH-independent, water-insoluble film former of the Eudragit RS type and is present at 10 to 40 percent by weight of the total composition

21. The composition of claim 20, wherein the ammonio methacrylate copolymer is Eudragit RS PO.

22. The composition of claim 17, wherein ethanol constitutes 50 to 80 percent by weight of the total composition and is the sole volatile solvent.7. The composition of claim 17, consisting essentially of nano-hydroxyapatite, an ammonio methacrylate copolymer, ethanol, and a flavoring oil.

23. The composition of claim 17, wherein the coating formed after drying has a dry thickness of 1 to 50 micrometers and remains adherent to oral hard tissues for at least 4 hours.

24. The composition of claim 17, wherein the nano-hydroxyapatite has a Ca/P molar ratio of 1.60 to 1.70 and crystallinity comparable to enamel apatite.

25. The composition of claim 17, wherein the composition is free of added fluoride compounds and free of rosin or colophony resins.

27. The composition of claim 17, wherein the ammonio methacrylate copolymer includes quaternary ammonium groups that impart a positive surface charge to the dried film to enhance adhesion to enamel and dentin.14. The composition of claim 1, packaged in a single-use applicator comprising a brush or foam tip preloaded with 0.1 to 1.0 mL of the composition.

28. The composition of claim 17, wherein the dried coating provides sustained calcium ion release of at least 0.05 mg per cm\hat{ }2 over 4 hours in simulated saliva.

29. Below are example patent claims (independent and dependent) drafted from the description you provided. These are illustrative claim formulations you can use or adapt for a patent application. They are not legal advice. 1. A method for remineralizing tooth enamel and reducing dentin hypersensitivity in a subject, comprising:
a) after a professional dental cleaning, drying exposed tooth surfaces;
b) applying to the dried tooth surfaces an amount effective to coat the dried tooth surfaces with a fluoride-free varnish composition comprising: nano-hydroxyapatite particles dispersed in ethyl alcohol and an ammonio methacrylate copolymer film-former;
c) allowing the ethyl alcohol to evaporate to form on the tooth surfaces a continuous adherent polymer film that retains the nano-hydroxyapatite;
d) maintaining the polymer film on the tooth surfaces for a dwell time sufficient to release calcium and phosphate ions from the nano-hydroxyapatite and to at least partially occlude dentin tubules. 2. The method of claim 1, wherein the nano-hydroxyapatite particles are nanoparticulate hydroxyapatite having an average particle diameter in the range of 10 nm to 500. nm. 3. The method of claim 1 or 2, wherein the varnish composition comprises from 0.5 to 40 wt % nano-hydroxyapatite based on total composition weight. 4. The method of any one of claims—3, wherein the ammonio methacrylate copolymer is present in the composition at a concentration of from 0.5 to 30 wt % based on total composition weight. 5. The method of any one of claims—4, wherein the varnish composition further comprises a flavoring oil. 6. The method of any one of claims—5, wherein the varnish composition is applied by brushing, swabbing, or by a dental applicator to provide a uniform coating. 7. The method of any one of claims—6, wherein the dwell time is from about 1 minute to about 48 hours, optionally from about 1 hour to about 24 hours. 8. A fluoride-free dental varnish composition, comprising nanoparticulate hydroxyapatite dispersed in an ethanol solution of an ammonio methacrylate copolymer film-former, wherein the ethanol is a volatile solvent that permits brushing application and rapid setting of the composition by evaporation, and wherein, upon application to cleaned tooth surfaces and drying, the composition forms a continuous adherent coating that releases calcium and phosphate ions to promote enamel remineralization and physically occludes dentin tubules. 9. The composition of claim 8, wherein the nanoparticulate hydroxyapatite has an average particle diameter in the range of 10 nm to 500 nm. 10. The composition of claim 8 or 9, wherein the nanoparticulate hydroxyapatite is present at 0.5 to 40 wt % of the composition. 11. The composition of any one of claims—10, wherein the ammonio methacrylate copolymer is present at 0.5 to 30 wt % of the composition. 12. The composition of any one of claims—11, further comprising a flavoring oil. 13. The composition of any one of claims—12, wherein the ethanol is present at 20 to 95 wt % of the composition. 14. A kit comprising: (a) the composition of any one of claims—13 in a container;
(b) written instructions for use instructing application to dried, professionally cleaned tooth surfaces and maintenance of the resultant polymer film for a dwell time effective to release calcium and phosphate ions and at least partially occlude dentin tubules. If you want, I can:—convert these into formal single-phrase claim format used in a particular jurisdiction,—expand dependent claims to include specific copolymer examples and trade names,—include specific ranges for film thickness, drying time, or clinical dwell times with supporting data.
